(19) United States
(12) Patent Application Publication
(10) Pub. No.: US 2006/0089539 A1
(43) Pub. Date: Apr. 27, 2006

(54) Integrated messages from multiple patient care devices

(76) Inventors: Saul Miodownik, West Hempstead, NY (US); Paul Frisch, Nanuet, NY (US); Paul Booth, New York, NY (US); Brian Stone, Brooklyn, NY (US)

Correspondence Address: Evans & Molinelli PLLC, 6411 Chapel View Road, Clifton, VA, 20124, US

(21) Appl. No.: 10/972,502

(22) Filed: Oct. 25, 2004

(51) Int. Cl. A61B 5/00; G06Q 10/00

(52) U.S. Cl. 600/300; 705/2

(57) ABSTRACT

Techniques for integrating messages from a medical care devices used to monitor or treat a patient include receiving association data. The association data indicates an association between a particular patient and one portable communication device assigned to one primary medical caregiver. The association data also indicates multiple associations between the patient and corresponding medical data generators. A medical data generator provides medical data to support an alert that indicates attention is desired from a medical caregiver. The medical data generators direct their data to diverse destinations. If an alert is issued based on any of the medical data generators, the portable communication device is determined based on the alert and the association data, a caregiver message is generated based on the alert, and the caregiver message is sent to the portable communication device. These techniques allow alerts from multiple medical devices to be integrated onto a single portable communication device.

### BACKGROUND OF THE INVENTION

1. Field of the Invention

The present invention relates to providing medical care needs of a patient in a hospital or other medical facility, and in particular to increasing medical facility efficiency in providing safe medical care by integrating alarms from multiple sources such as devices to monitor or treat the patient, or both.

2. Description of the Related Art

Hospitals, clinics, medical offices and other medical facilities of healthcare organizations (also called healthcare facilities) are in the business of delivering medical care to patients. Such facilities stay in business by meeting standards promulgated by certifying bodies for patient care and by successfully competing with other hospitals in providing quality medical care to patients. One measure of quality of care is patient safety. Patient safety depends on careful monitoring of patient condition, accurate application of patient treatment regimes, including therapy and medication, correct response to changes in patient condition, and timely response to changes in patient condition.

For example, The Joint Commission for Accreditation of Healthcare Organizations (JCAHO) examines aspects of patient safety in healthcare organizations. The recently promulgated JCAHO 2004 National patient safety goals include: 1] improvement in the effectiveness of clinical alarm systems (including enhanced alarm management and improved alarm communication and response); 2] improvement in the accuracy of patient identification; 3] improvement in the safety of using high-alert medications; 4] improvement in the safety of using infusion pumps; and, 5] improvement in the effectiveness of communication among caregivers. Caregivers include physicians, nurses, therapists, technicians and others who proved care to patients in a medical facility.

Clinical alarms are generated by many known communication systems, patient status monitoring systems and patient care delivery systems. Communications systems include, but are not limited to, nurse call buttons, toilet emergency buttons, caregiver locators, intercoms, beepers, pagers, beside controls, wired and wireless telephones, and personal digital assistants (PDAs). Monitoring systems include, but are not limited to, vital signs monitors for a variety of indicators of patient conditions, such as body temperature, heart rate, blood pressure, and blood oxygen saturation level. Patient care delivery systems include, but are not limited to, feeding tubes, intravenous (IV) tubes, fluid reservoir bags, medication containers, infusion pumps, lung ventilators, dialysis units and bodily waste collection bags. Infusion pumps and controllers are used extensively for delivering intravenous fluids and medications as part of the care of patients in hospital. It is not uncommon for three or more infusion pumps to be used on the same patient.

Currently, diverse alarms are provided with different systems. For example, a bedside nurse call button causes a light to turn on over the doorway of a patient's room. A code blue alert is triggered by a caregiver in a room when a life-threatening emergency is perceived and causes a signal to be transmitted to a central nursing station. A problem with an infusion pump, such as a pressure buildup indicating blockage downstream of the pump, or a pressure drop indicating an empty fluid supply, may cause the infusion pump to issue an alarm, such as a distinctive audible signal using a bell, buzzer, whistle or other sound generator. Similarly, a problem detected by a lung ventilator may result in the ventilator issuing an alarm. Some medical care devices are designed as part of a system to send an alarm by way of a radio signal to a personal communication device worn by a caregiver, such as a system-specific or general-purpose pager. Some medical devices are designed as part of a system to be hooked up to a computer or network to send data, automatically or in response to polling, to a system-specific computer program (e.g., a system-specific server executing on a computer linked to the network). The computer program processes the data to determine status of the medical device, including alarm conditions, and displays the status on some display, typically a video screen. The caregiver views the display to determine the status and then decides on a course of action.

As a result of diverse alarms, a caregiver may be encumbered by too many personal communication devices and inundated with too many alarms to process at one time. For example, in a typical configuration a nurse carries a hospital pager, a DATA CRITICAL™ physiologic monitoring system pager f from GE Healthcare Technologies, Milwaukee, Wis., a ventilator management system manufactured by Cardiopulmonary Associates, Milford, Conn., a nurse call pager, a hospital pager, a cell phone, and a nurse locator infrared tag from HillROM of Batesville, Ind. This configuration encumbers a nurse with multiple different personal communication devices. When multiple devices signal for the nurse's attention simultaneously the nurse might not be able to respond to all at once. Alarms that fire too frequently are often ignored. Alarms that require the caregiver to poll a particular location in the facility or on the network might be overlooked while the caregiver attends to alarms that have been passed to a personal communication device carried with the caregiver.

Alarms that display at a central site have to be forwarded to the caregiver associated with a patient. There are often staffing assignment tools that associate caregivers to patients and there is typically an Admission/Discharge/Transfer (ADT) system that identifies where a patient is within the facility. The medical devices that generate the alarms used to monitor and treat the patient are typically portable systems that are moved into the patient's room as needed. To forward an alarm to the assigned caregiver, an attendant at the central site typically uses an at least partly tedious and error-prone manual process to identify the room where the alarm is generated, to identify the patient in that room based on the ADT system, to identify the caregiver assigned to that patient based on a separate staffing system, to find a communication device address or phone number to use to contact the caregiver based on a separate contact system, to composes a free text or code message, and to send the message to the caregiver using the identified communication device.

As a consequence, the alarm formats for multiple alarms issued by multiple medical care devices have some impact on the sequence in which the associated conditions are attended. In many cases, the sequence followed is not the optimal or prudent sequence in terms of patient care or safety.

High-alert medications are drugs that have a high risk of causing injury when they are misused. Consequences of errors may be devastating; therefore, these medications are often packaged differently, stored differently, prescribed differently, and administered differently than others. Methods should be developed and instituted that make it near impossible for the drug to be given in a potentially lethal manner. At the time of this writing high-alert medications that are administered through an IV tube include, but are not limited to, amiodarone, heparin, insulin, and lidocaine. Many current systems control the administration of medications, including high-alert medications, with manual procedures that are tedious and error prone. For example, in some facilities, the caregiver administering the medication also enters an identification code for the medication and other information manually onto a keyboard or onto a sheet of paper for later transcription to a computer by a second employee of the medical facility.

The response to an alarm often requires the caregiver to know something about the history of treatment for a patient. An alarm raised because of severe symptoms by a patient might normally involve the administration of some medication. However, depending on when that medication or a conflicting medication was last administered by the same or a different caregiver, the appropriate response might be different. As another example, an alarm indicating an empty glucose solution supply bag on one infusion pump might be ignored for a time as a non-critical situation. However, if the patient is also receiving insulin administered by a different infusion pump, providing a replacement bag might be more critical. Therefore, the caregiver often needs to access the medical history of the patient. Insofar as that history is unavailable, incomplete or inaccurate, the wrong response to the alarm may result.

Often there are multiple caregivers at different times and with different levels of responsibility in caring for the same patient. In addition, a caregiver is usually responsible for many different patients. Insofar as the proper caregiver is not advised of the alarm or the caregiver is confused as to the patient who is affected by the alarm, the wrong response to the alarm may result.

Based on the foregoing, there is a clear need for a system to integrate messages from multiple devices used in the care and treatment of patients in a medical facility and that does not suffer from the deficiencies of prior art approaches.

### SUMMARY OF THE INVENTION

Techniques are provided for integrating messages from a plurality of medical care devices used to monitor or treat a patient. These techniques allow alerts from diverse medical devices to be prioritized, filtered and integrated onto a single portable communication device in the possession of a caregiver.

In a first set of embodiments, a method includes receiving association data. The association data indicates an association between a particular patient and one portable communication device assigned to one primary medical caregiver. The association data also indicates multiple associations between the patient and corresponding medical data generators. A medical data generator provides medical data to support an alert that indicates attention is desired from a medical caregiver. The multiple medical data generators direct medical data to two or more different destinations. If it is determined that an alert is issued based on any of the medical data generators, the portable communication device is determined based on the alert and the association data, a caregiver message is generated based on the alert, and the caregiver message is sent to the portable communication device.

In some embodiments of the first set, the plurality of medical data generators includes a device for human communication, a device for monitoring a condition of a patient, or a device for delivering a treatment to a patient, or some combination. In some of these embodiments, the device for human communication is a manually operated switch that generates an alert that indicates at least one of a nurse call, an emergency call, and a code blue call. In some of these embodiments, the device for monitoring a condition of a patient is a vital signs monitor that generates an alert that includes vital signs data. In some of these embodiments, the device for delivering a treatment to a patient is an infusion pump that generates an alert that includes pump operation status. In some of these embodiments, the device for delivering a treatment to a patient is a lung ventilator that generates an alert that includes ventilator operation status.

In some embodiments with an infusion pump, the association data further indicates an association between the particular patient and a medication being administered to the particular patient by the infusion pump. In these embodiments, dose data that indicates the dose of the medication being administered is determined based on pump operation status in the alert and association data that indicates the medication. The caregiver message is generated based in part on the dose data.

In some embodiments of the first set, medical records data that indicates medical history of the particular patient are also received and the caregiver message is generated based in part on the medical records data. 100181 In some embodiments of the first set, generating the caregiver message includes determining a degree of importance for the alert and basing the caregiver message in part on this degree of importance, thus prioritizing the alert in the caregiver message. In some of these embodiments, generating the caregiver message also includes determining whether the alert warrants a caregiver message based on the degree of importance. The caregiver message is only sent if it is determined that the alert warrants one, thus filtering the alerts received from the medical data generators.

In some embodiments of the first set, receiving association data includes receiving first data and second data from a reader that senses an identification structure. In some of these embodiments the identification structure is a bar code marking. In some of these embodiments the identification structure is a radio frequency identification tag (RFID tag). In various of these embodiments, an identification structure is connected to the primary caregiver, the patient, the multiple medical data generators, and a container holding medication administered to the patient.

01 In some embodiments of the first set, a particular device of the medical data generators is capable of delivering a treatment to a patient. The method includes determining a particular treatment to be performed by the particular device based at least in part on the received alert. A treatment message that indicates the particular treatment is sent to the particular device, which causes the particular device to deliver the particular treatment to the patient. In some of these embodiments, the particular treatment is determined based on receiving from the portable communications device an instructions message that indicates information entered by the primary medical caregiver for providing treatment to the patient.

In some embodiments of the first set, the association data includes a time when each association began. In some of these embodiments, each association in the association data is stored on a nonvolatile storage medium without removing association data previously stored on the storage medium, thus producing an audit trail for care given to the patient.

In some embodiments of the first set, the association data further indicates an association between the patient and a particular location in a medical care facility. The particular location is stored in association with each of the medical data generators, thus providing a means to locate medical data generators in the medical care facility.

In other sets of embodiments, systems and computer readable media implement the methods described above.

In another set of embodiments, an apparatus for reporting human communications regarding the condition or treatment of a patient includes a network interface, multiple switches, and a sound sensor. The network interface is coupled to a network for communicating a data packet. The switches are used for indicating multiple conditions in the vicinity of a patient. The apparatus also includes one or more circuit blocks configured to detect closure of a first switch In response to detecting closure of the first switch, a first data packet that indicates a condition associated with the first switch is sent to the network through the network interface. The circuit blocks also forming digital sound data from sound sensed by the sound sensor and form a stream of data packets based on the digital sound data using a protocol for transferring sound over the network. The stream of data packets are sent to the network through the network interface.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 is a block diagram that illustrates a system in a medical facility that integrates messages from multiple diverse systems, according to an embodiment;

FIG. 2A is a block diagram that illustrates multiple network message sources for a system in a medical facility, according to an embodiment;

FIG. 2B is a block diagram that illustrates details of a patient station network message source, according to an embodiment;

FIG. 3A is a flow diagram that illustrates at a high level a method for integrating messages from multiple diverse systems, according to an embodiment;

FIG. 3B is a flow diagram that illustrates in more detail a step in the method of FIG. 3A for generating association data, according to an embodiment;

FIG. 4A is a flow diagram that illustrates at a high level a method for integrating messages from multiple diverse systems at a server on the network, according to an embodiment;

FIG. 4B is a flow diagram that illustrates in more detail a step in the method of FIG. 4A for receiving association data, according to an embodiment;

FIG. 4C is a block diagram that illustrates an association table for storing or archiving association data, according to an embodiment;

FIG. 5 is a block diagram that illustrates a system for integrating medical messages from multiple diverse systems, according to a more detailed embodiment;

FIG. 6 is a block diagram that illustrates a computer system upon which an embodiment of the invention may be implemented.

### DETAILED DESCRIPTION

A method and system are described for integrating messages from a plurality of medical care devices used to monitor or treat a patient. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

In the following, several embodiments are described in the context of client and server processes operating on an inter-network of local area networks. However, the invention is not limited to this context and can be embodied using a single local area network or other kinds of communication systems using various combinations of hardware and software to implement the methods described herein.

1. Structural Overview

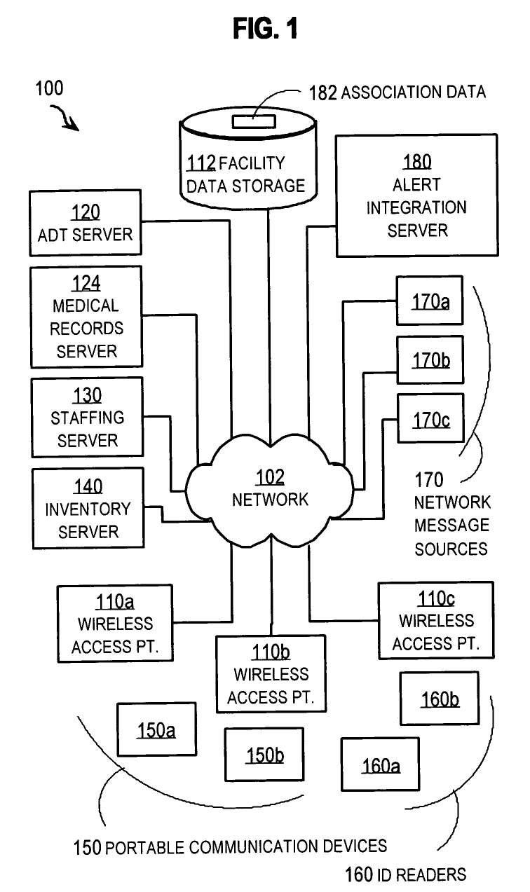

FIG. 1 is a block diagram that illustrates a system 100 in a medical facility that integrates messages from multiple diverse systems, according to an embodiment. The system 100 includes a network 102, facility data servers 120, 124, 130, 140, facility data storage 112, network message sources 170, wireless access points 110a, 110b, 110c (collectively referenced herein as wireless access points 110), portable communication devices 150, identification (ID) readers 160, and an alert integration server 180.

The client-server model of computer process interaction is widely known and used. According to the client-server model, a client process sends a message including a request to a server process; and the server process responds by providing a service. The server process may also return a message with a response to the client process. Often the client process and server process execute on different computer devices, called hosts, and communicate via a network using one or more protocols for network communications. The term “server” is conventionally used to refer to the process that provides the service, or the host computer on which the process operates. Similarly, the term “client” is conventionally used to refer to the process that makes the request, or the host computer on which the process operates. As used herein, the terms “client” and “server” refer to the processes, rather than the host computers, unless otherwise clear from the context. In addition, the process performed by a server can be broken up to run as multiple servers on multiple hosts (sometimes called tiers) for reasons that include reliability, scalability, and redundancy, but not limited to those reasons. In some embodiments multiple servers and clients execute on the same host.

The network 102 is any network that connects a variety of users of host computers, including, but not limited to, local area networks (LANs), wireless networks, wide-area networks (WAN), the Internet (a network of heterogeneous networks using the Internet Protocol, IP), and virtual private networks. In an illustrated embodiment, network 102 is an inter-network of LANs within a medical facility communicating using the Transport Control Protocol (TCP) within the Internet Protocol (IP), a combination well known as TCP/IP. An advantage of TCP/IP is that data transmitted using TCP/IP can make the leap from one LAN to another across one or more network devices, such as bridges and routers. Network 102 includes zero or more hosts (not shown) that perform as terminals for various caregivers to access the network and devices in communication with the network. Various facility personnel, including administrators, doctors, nurses, pharmacists and other staff members, use these terminals.

The wireless access points 110 are network devices that support wireless communications with multiple nodes using a wireless protocol such as IEEE 802.11, BlueTooth, and cellular telephone protocols, well known in the art. In the illustrated embodiment the wireless access points 110 communicate with one or more portable communications devices 150, including portable communication devices 150a, 150b, and one or more ID readers 160, including ID readers 160a, 160b, to which a population of caregivers have access. In some embodiments, one or more of the portable communications devices 150 are cellular phones. In some embodiments, one or more of the portable communications devices 150 are personal digital assistants (PDAs) well known in the art.

ID readers 160 are devices that can determine an identification value associated with an identification structure attached to some item. Any ID readers may be used. In the illustrated embodiments, the ID readers 160 are bar code scanners for reading bar code markings on an item In other embodiments, the ID readers are RFID readers for detecting an RFID tag attached to the item. In the illustrated embodiments, one or more of the ID readers 160 are wireless readers communicating over network 102 through wireless access points 110. In other embodiments, one or more of the ID readers 160 are connected to network 102 by wires or cables (not shown).

For the purposes of illustration, three wireless access points 1110a, 110b, 110c and two portable communications devices 150a, 150b and two ID readers 160a, 160b are shown in FIG. 1. In other embodiments more or fewer wireless access points 110 are connected to network 102 for communication with more or fewer portable communications devices 150 and more or fewer ID readers 160.

The system 100 includes four heritage medical facility systems, including an Admission/Discharge/Transfer server (ADT server) 120, a patient medical records server 124, a staffing server 130, and one or more inventory servers 140 that store data on one or more devices that provide facility data storage 112. The heritage medical facility systems perform functions of data management that exist at the medical facility, or are commercially available, when an embodiment of the present invention is implemented for the facility. Any known systems may be used for the heritage medical facility systems. In the illustrated embodiment, the ADT server 120 manages data related to the admission, transfer and discharge of a patient in the medical facility so that the status and location of the patient in the facility is tracked. The patient medical records server 124 manages data related to the history of the patients health, including previous and present assessments, diagnoses, prescribed medications and other treatments. The staffing server 130 manages data related to the facility staff and includes information about caregivers, including contact numbers for cell phones, pagers, or PDAs assigned to the caregivers, shifts worked and scheduled to be worked, supervisory hierarchies, and patient assignments. The inventory server 140 manages data related to the equipment and supplies obtained by the medical facility, including equipment type, serial number, inventory control number and, for fixed equipment, location. In various embodiments, system 100 includes more or fewer heritage medical facility systems like those that include servers 120, 124, 130, 140.

The heritage medical facility systems store facility data on one or more storage devices indicated in FIG. 1 by facility data storage 112. The servers 120, 124, 130, 140 control the storage and retrieval of data. For purposes of illustration, one facility data storage 112 is shown connected to network 102, but in other embodiments, the data may be distributed over several data storage devices connected directly to one or servers, or connected indirectly to one or more servers through network 102. Any facility data storage known in the art may be used as facility storage 112.

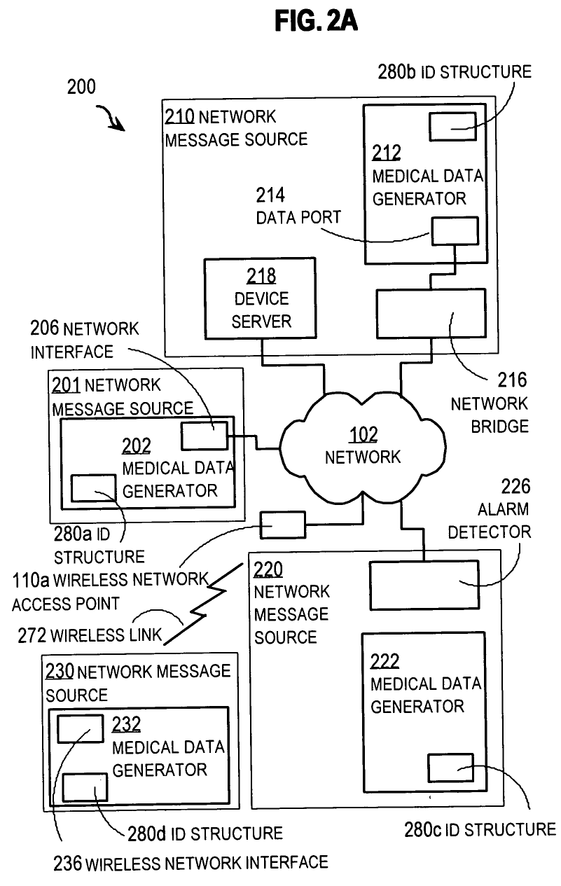

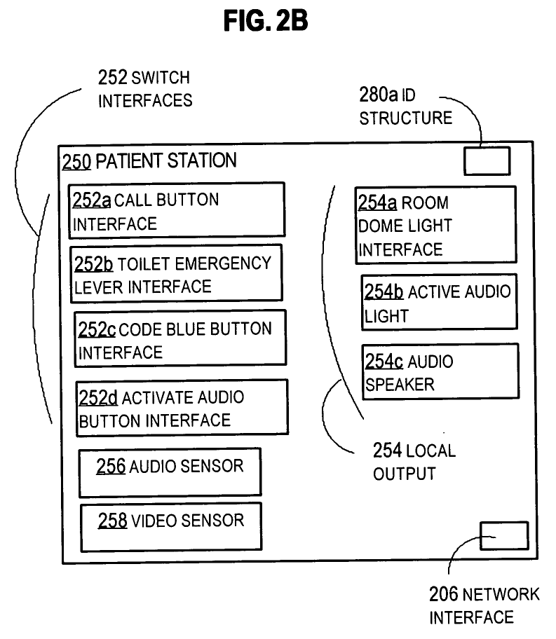

The network message sources 170, including network message sources 170a, 170b, 170c, generate network-communicated messages that indicate the current condition or treatment of a patient. The data in these messages is generated at least in part by medical data generators used to monitor or treat patients in the medical facility. Some network message sources 170 are also medical data generators, such as the DATA CRITICAL vital signs monitoring system, GE Healthcare Systems physiological monitoring, and the Cardiopulmonary Associates “Bernoulli” Ventilator management system. The GE Healthcare physiological monitoring systems reports vital signs data on a network and records the data and transmits messages when attention is required. The vital signs data may be queried by other applications through the network to determine this information. When attention is required a particular communications device receives an alert. A caregiver holding the particular communications device then responds. Some message sources 170 are assembled by combining medical data generators that do not use network 102 with network devices that place messages onto the network 102, as described in greater detail below with reference to FIG. 2.

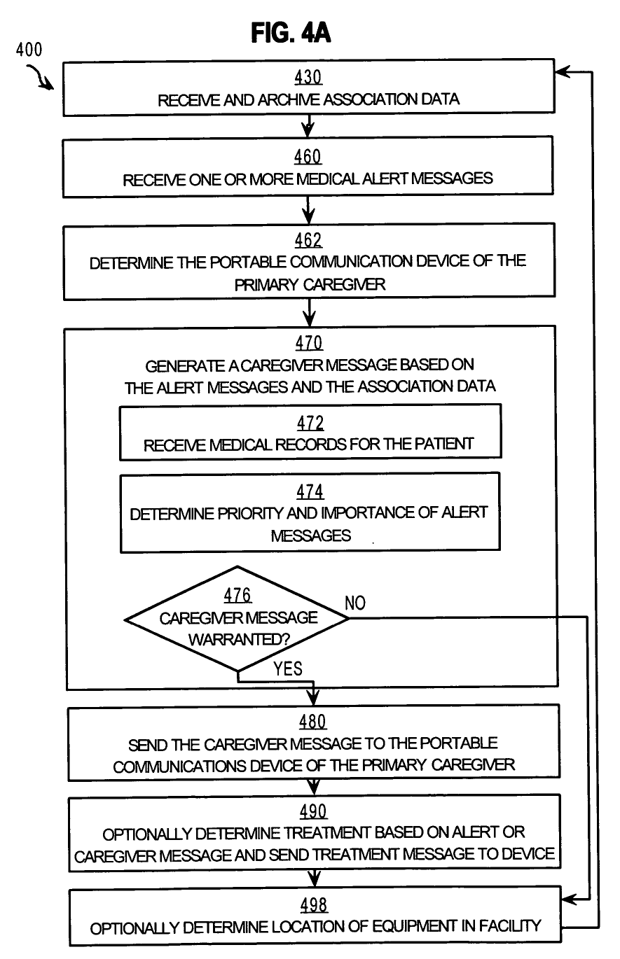

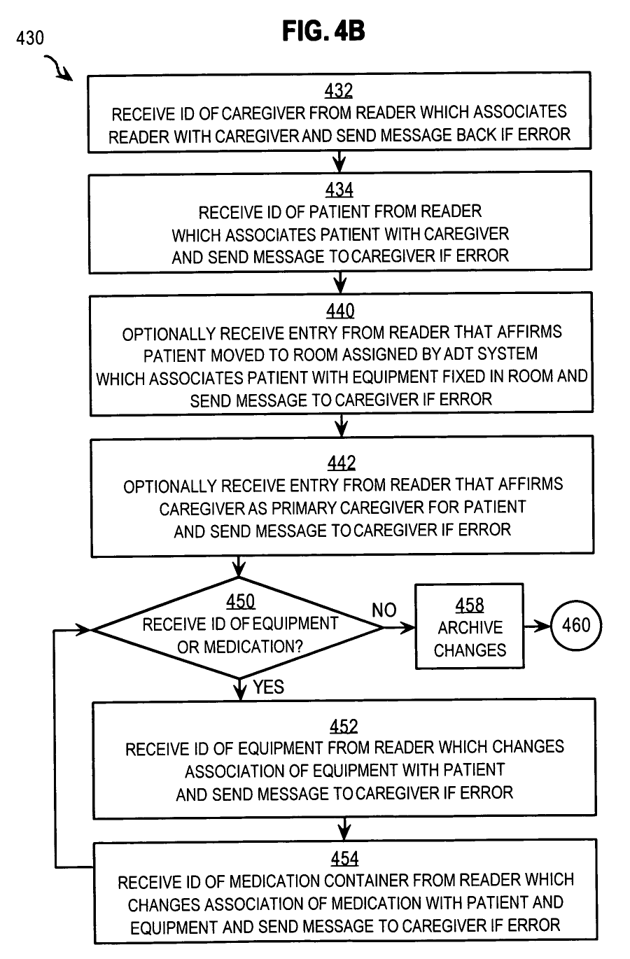

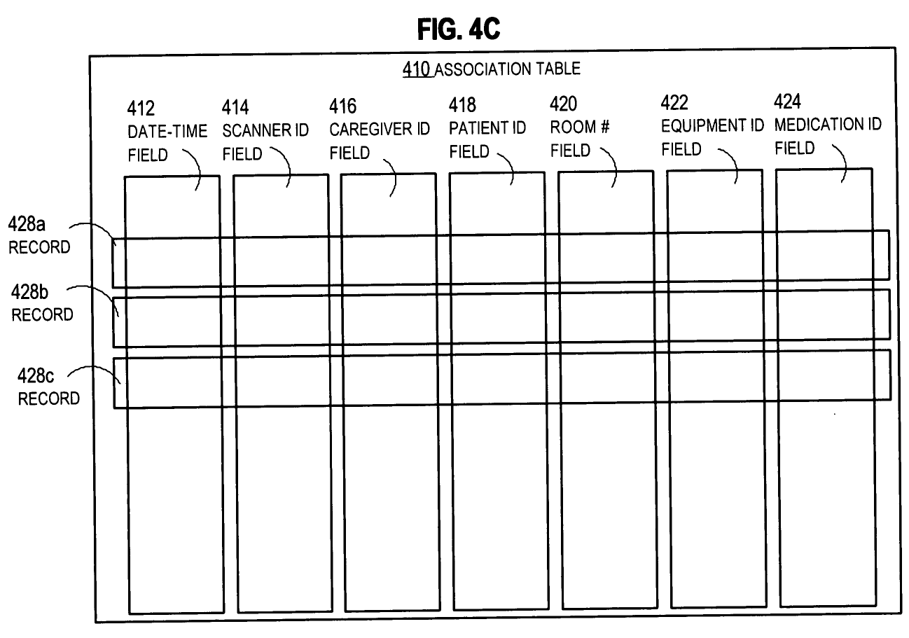

The alert integration server 180 is a process that combines information from any of the network message sources 170. The alert integration server 180 manages association data 182, which is stored on facility data storage 112 in an illustrated embodiment. The association data indicates associations between caregiver, patient and medical data generators for network message sources 170. The processes performed by alert integration server 180 are described in the next section and in more detail below with reference to FIGS. 4A and 4B. The association data 182 is described in more detail below with reference to FIG. 4C. In some embodiments, one or more functions described above for heritage medical facility systems, including servers 120, 124, 130, 140, are performed by the alert integration server 180.

2. Functional Overview

According to illustrated embodiments, multiple medical data generators used to monitor or treat a multiple patient s are configured as message sources 170 to send messages on network 102. The alert integration server 180 combines information from any of the message sources 170 associated with patients for which any caregiver of multiple caregivers is primarily responsible and sends a message, called a caregiver message, to that particular caregiver over a single portable communication device, e.g. 150a. Thus each caregiver is freed from carrying multiple communication devices for multiple diverse systems. In some embodiments, the alert integration server 180 also prioritizes and filters messages from one or more message sources that generate messages that occur within a short time, thus assisting the caregiver in dealing with multiple competing calls for attention. In some embodiments, the caregiver also responds through the single portable communication device to deliver treatment to a patient by sending a message to a medical data generator configured to deliver that treatment.

3. Network Message Sources

FIG. 2A is a block diagram that illustrates details for multiple network message sources in a system 200, according to an embodiment. A medical message source generates medical alert messages on network 102 that indicate that attention is desired from a medical caregiver based on data from a medical data generator that supports such an alert. The system 200 includes the network 102 and wireless access point 110a described above, and four particular network message sources 201, 210, 220, 230 that each may serve as any of the message sources 170 depicted in FIG. 1.

Network message source 201 includes a medical data generator 202 that is connected directly to network 102 via a network interface card 206. For example, a scheduled event reminder system is connected to the network 102 via network interface 206. The scheduled event reminder system sends a message to a caregiver when it is time to perform a scheduled procedure, such as administering a medication.

Another example of a medical data generator 202 that is connected directly to network 102 via a network interface card 206 is a digital patient station. The patient or a person at the patient's bedside uses a patient station to report on the patient's condition or desire for attention from a caregiver, or both. FIG. 2B is a block diagram that illustrates details of a digital patient station 250 as a network message source, according to an embodiment.

In the illustrated embodiment, the patient station 250 includes interfaces 252 for switches (e.g., buttons and levers), an audio sensor 256, a video sensor 258 and local output 254, in addition to the network interface 206. The switch interfaces 252 include a call button interface 252a, a toilet emergency interface 252b, a code blue button interface 252c, and an activate-audio button interface 252d. When a person operates a nurse call button, the action is detected by the call button interface 252a and a message that indicates this action is sent through network interface 206 to the network 102 and then to alert integration server 180. When a person operates a toilet emergency lever, the action is detected by the toilet emergency lever interface 252b and a message that indicates this action is sent through network interface 206 to the network 102. When a person operates a code blue button, the action is detected by the code blue button interface 252c and a message that indicates this action is sent through network interface 206 to the network 102.

When a person operates an activate-audio button, the action is detected by the activate-audio button interface 252d, the audio sensor 256 is activated, and the person's speech is transmitted through network interface 206 over network 102 using an appropriate protocol, such as voice over IP (VoIP). In some embodiments, the audio sensor 256 and VoIP transmission is activated based on a message received at the patient station 250 from the network 102.

The local output 254 includes a room dome light interface 254a, an active audio light 254b and an audio speaker 254c. The room dome light interface 254 controls a beacon over a doorway to a room where the patient is located. Typically, the beacon emits a visible or audible signal, or both, that can be perceived in a hallway or other passageway leading to a room where the patient is located. In some embodiments, 245a is omitted and a signal is sent to the beacon through network interface 206. The active audio light 254b emits light when a person operates the activate-audio button as detected by the activate-audio button interface 252d, and the person's speech is transmitted through network interface 206 over network 102. Light from the active audio light 254b warns a person, in the interests of privacy, that voice data is being transmitted from the patient station over the network. The audio speaker 254c generates sounds at the patient station 250, such as voice data received through network interface 206 from network 102 using VoIP, and locally generated ringing, whistling or buzzing sounds to indicate that the toilet emergency or code blue switches have been closed.

In the illustrated embodiment, the patient station 250 includes a video sensor, such as a video camera, to capture one or more images that depict conditions in the vicinity of the patient. Video data is sent as digital video data through network interface 206 to the network 102. In some embodiments, the video sensor 258 is replaced by an interface to a separate video camera.

In other embodiments, a patient station includes more or fewer switch interfaces 252, local output 254 and sensors 246, 258. For example, a video monitor patient station includes only the video sensor 268 and the network interface 206 and an active video light (not shown). More than one patient station may be fixed in the vicinity of the same patient. For example, a patient station without video sensor 258 is fixed in a patient room along with a video monitor patient station described above.

Referring again to FIG. 2A, the medical data generator 202 includes an attached ID structure 280a that can be read by an ID reader 160. Any ID structure known when an embodiment is implemented can be used, including but not limited to a bar code marking and a RFID tag. An ID structure 280a is attached to a mobile medical data generator that can be moved from one location in the facility to another in order to inventory equipment in the medical facility more easily. As described in more detail below, the ID structure 280a is also used to associate a data generator with a particular patient quickly and easily. In some embodiments in which the medical generator 202 is fixed at a location in a medical facility, the ID structure 280a is omitted. For example, in some embodiments, the ID structure 280a is omitted from a patient station 250 fixed at a location in a patient's room in the medical facility. An advantage of including an ID structure 280a on a mobile medical data generator is that the generator can be tracked easily as it is moved form one patient to another

Network message source 210 includes a medical data generator 212 that is not connected directly to the network 102. Network data generator 212 includes a data port 214 for sending data to an application, such as device server 218, running on a separate computer, but does not include a network interface card. For example, a Bernouilli Ventilator includes an RS232 data port, well known in the art, for passing data back and forth with a computer. In the illustrated embodiment, the medical data generator 212 is configured as a network message source 210 by connecting the data port to a network bridge 216. The network bridge converts between data streams configured for data port 214, such as RS232 data streams, and a network protocol, such as TCP/IP, used to transfer data on network 102. In some embodiments, the network bridge 216 is commercially available for specific data ports and network protocols. In some embodiments, network bridge 216 is a general purpose computer running software to perform the conversion.

In the illustrated embodiment, medical data generator 212 is controlled by device server 218 to deliver treatment, send data, and indicate situations that call for caregiver attention. In some embodiments, the device server 218 executes on a host computer connected directly to data port 214. In the illustrated embodiment, the device server 218 is connected to the network 102 to control multiple medical data generators, such as Bernoulli ventilators used on several patients. The device server 218 generates messages that indicate caregiver attention is desired based on data received from medical data generator 212 over network 102. Therefore, the device server 218 is included in the configuration to perform as a network message source 210 to generate medical alert messages that indicate attention is desired from a medical caregiver.

In the illustrated embodiment, the medical data generator 212 includes an attached ID structure 280b that can be read by an ID reader 160.

Network message source 220 includes a medical data generator 222 that is not connected directly to the network 102. Network data generator 222 does not include a data port for sending data to an application. Some heritage medical devices monitor or treat patients without digital data output. Such devices qualify as medical data generators if they emit a signal that can be used to support an alert message for attention from a medical caregiver. For example, some early model infusion pumps emit an acoustic signal to indicate a problem, such as a dry supply line. To provide a network message based on such signals, an alarm detector 226 is included in network source 220. Alarm detector 226 detects a non-digital signal emitted from medical data generator 222 and produces a message in a protocol, such as TCP/IP, suitable for transmission over network 102 to alarm integration server 180.

In the illustrated embodiment, the medical data generator 222 includes an attached ID structure 280c that can be read by an ID reader 160.

Network message source 230 includes a medical data generator 232 that is connected directly to network 102 via a wireless link 272 to wireless access point 110a using a wireless network interface 236. For example, a recent model infusion pump system is connected to the network 102 using wireless network interface 236. Devices that adapt medical data generators for network communications, such as network bridge 216 and alarm detector 226 in network message sources 210, 220, respectively, are called herein “network adapters.” In some embodiments, one or more of network adapters 216, 226 in network message sources 210, 220, respectively, are connected to network 102 using a wireless network interface like interface 236.

In the illustrated embodiment, the medical data generator 232 includes an attached ID structure 280d that can be read by an ID reader 160

4. Method for Integrating Messages from Multiple Diverse Systems

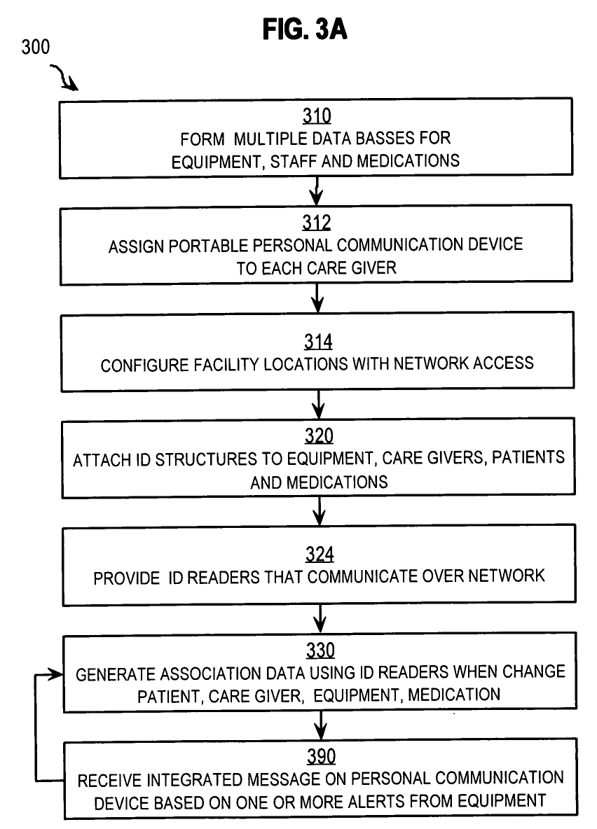

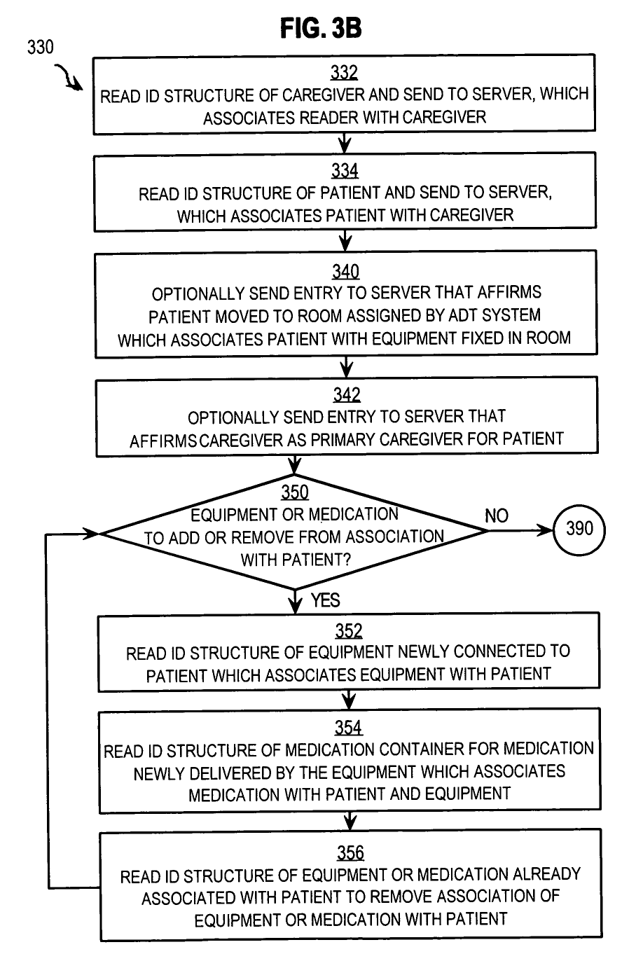

FIG. 3A is a flow diagram that illustrates at a high level a method 300 for integrating messages from multiple diverse systems, according to an embodiment. Although steps are depicted in FIG. 3A and the following flow diagrams in a particular order for purposes of illustration, in other embodiments one or more steps may be performed in a different order, or overlap in time, or be omitted, or changed in some combination of order, overlap and omission.

In step 310, multiple data bases are formed to record information related to patients, staff, equipment and medications. In embodiments in which previously established heritage systems that include ADT server 120, medical records server 124, staffing server 130, and inventory server 140 are maintained, step 310 may be omitted.

In step 312 a portable personal communications device is assigned to each caregiver. For example, each caregiver is assigned a hospital pager or cellular telephone and that assignment is recorded using the staffing server by adding the pager dialup number to the records related to each caregiver. In an illustrated embodiment, each caregiver is assigned a PDA and the unique media access code (MAC) number manufactured into each PDA is added to the data managed by the staffing server. For example, nurse Jane Doe is assigned a PDA with a MAC address, designated herein for convenience by the characters “MAC 1.” The MAC address MAC1 is recorded as the PDA for nurse Doe in the staffing data managed by staffing server 130.

In step 314, locations in the facility are configured with network access. For example, network junction boxes are wired into each room, and multiple wireless access points 110 are distributed throughout the facility. Such configurations can be accomplished by contracting for commercial services currently available and widely known in the art. Alternatively, medical facility personnel trained in information technology can accomplish or contribute to the effort. In some embodiments, step 314 includes obtaining network adapters for medical data generators that do not include network interfaces. In some embodiments, step 314 includes wheeling mobile equipment into a room and plugging a network cable from a network adapter for the equipment into a network junction box in the room. In some embodiments, at least some of the mobile equipment is configured with a wireless network interface or a wireless network adapter.

In step 320, ID structures are attached to equipment, caregivers, patients and medications. In an illustrated embodiment, bar code markings are taped to equipment, included in badges worn by caregivers, printed on wristbands worn by patients, and included on packages of purchased medication or printed by pharmacy staff of the medical facility when medications are repackaged in containers, such as fluid supply bags, for use in the medical facility. In some embodiments, one, several or all of these bar marking are replaced by RFID tags.

In step 324, ID readers that communicate over the network are provided to the caregivers. For example, a stock of wireless bar code scanners is kept at a central station in the medical facility. A caregiver can obtain one wireless bar code scanner at the beginning of the caregiver's shift from the stock to use during that shift and return at the end of the shift. For example, nurse Doe checks out a bar code scanner 160b with MAC address, designated herein for convenience as “MAC2.” In other embodiments, one or more wireless RFID readers or wired scanners or wired RFID readers are made available. In some embodiments, a wireless ID reader is included in the PDA assigned to a caregiver.

In step 330, the caregiver generates association data using the ID readers whenever the caregiver administers a change to the patient location, equipment or medication. Association data indicates an association between a particular patient and a portable communication device assigned to a primary medical caregiver. In some embodiments, association data that associates a caregiver as the primary caregiver for a patient is based on an assignment retrieved from the staffing server 130. Association data also indicates multiple associations between the patient and multiple medical data generators. A medical data generator is a device used to monitor or treat a patient, which produces data that supports a medical alert message to get the attention of a caregiver. For example, nurse Doe generates association data for patient John Smith using the ID reader 160b. More detailed steps on generating the association data are described below with reference to FIG. 3B.

In step 390, a primary caregiver receives an integrated message on the assigned portable personal communication device based on one or more alert messages supported by data from one or medical data generators among the equipment. For example, nurse Doe receives a message on her PDA, with MAC address MAC1, when a medical data generator used with patient Smith supports a medical alert message sent on network 102.

Control passes back and forth between steps 390 and 330 as the primary caregiver moves, as dictated by a caregiver duty cycle, between receiving calls for attention in step 390 and executing changes in patient care in step 330.

FIG. 3B is a flow diagram that illustrates in more detail step 330 from method 300, for generating association data, according to an embodiment.

In step 332, an ID reader is used by a caregiver to read an ID structure for the caregiver and send the ID to the alarm integration server 180. The server 180 uses this information to associate the caregiver with the ID reader, as described in more detail in the next section. For example, nurse Jane Doe checks out ID reader 160b, a bar code scanner with MAC address MAC2, at the beginning of her shift by scanning her ID badge with the ID reader 160b. As a result a message is sent over network 102 to alarm integration server 180 that ID reader 160b is associated with caregiver Jane Doe. The ID reader 160b is identified by its MAC address, MAC2, included in the TCP/IP message. Nurse Doe is identified by her employee ID number represented in the bar code on her badge, designated herein for convenience as CIDDOE. This association remains until a different caregiver ID is scanned by ID reader 160b.

In step 334, the ID reader is used by the caregiver to read an ID structure for the patient and send the ID to the alarm integration server 180. The server 180 uses this information to associate the patient with the caregiver, as described in more detail in the next section. For example, nurse Jane Doe scans the bar code on the wristband of patient Smith. Smith's patient ID number represented in the bar code on his wristband is designated herein for convenience as PIDSMITH. As a result, a message is sent over network 102 to alarm integration server 180 that ID reader 160b is associated with PIDSMITH. This also associates the caregiver CIDDOE who is using scanner 160b with patient PIDSMITH. This also indirectly associates the PDA stored with the staffing records for caregiver CIDDOE, which PDA has a MAC address MAC1, with patient PIDSMITH.

In step 340, the ID reader is used by the caregiver to affirm that a patient has been moved to a room as assigned by the ADT server, if the patient is moved there by the current caregiver. If the patient is already in the assigned room, step 340 is omitted. Any method may be used to affirm this. In some embodiments, nurse Doe scans a bar code attached to a fixed item in the room, such as a doorjamb or a patient station, if nurse Doe brings patient Smith to that room. For example, if nurse Doe brings patient Smith into room 222 which has a location ID designated herein for convenience as LID222, she scans the bar code on the doorjamb. As a result, a message is sent over network 102 to alarm integration server 180 that ID reader 160a is associated with LID222. This causes the server 180 to associate PIDSMITH with LID222.

In step 342, the ID reader is used by the caregiver to affirm that the caregiver is the primary caregiver for patient Smith. If the primary caregiver is not changing, step 342 is omitted. Any method may be used to affirm the primary caregiver. In some embodiments, nurse Doe punches a button on the bar code reader to affirm a change in primary caregiver. As a result, a message is sent over network 102 to alarm integration server 180 that ID reader 160a is in the hands of the primary caregiver. This causes the server 180 to associate CIDDOE as the primary caregiver for PIDSMITH. In other embodiments, nurse Doe is identified as the primary caregiver for patient Smith on the basis of data managed by the staffing server 130, and step 342 is omitted.

In step 350 it is determined if there are any changes to the equipment or medication for the patient. If not, control passes out of step 330 to step 390 shown in FIG. 3A, and described above. For example, after identifying herself as the primary caregiver for patient Smith in step 342, nurse Doe checks the equipment and medication and sees that all is in order and leaves the room. She then associates herself with the next patient and tends to the that patient as reasonable. Alternatively, she receives and responds to a message based on the equipment or medication involved with patient Smith or other of her patients.

If there are any changes to the equipment or medication for the patient, control passes to step 352. In step 352, the ID reader is used by the caregiver to read an ID structure attached to equipment that the caregiver is currently attaching to the patient. The server 180 uses this information to associate the equipment with the patient whose ID structure is most recently read, at least for equipment such as data generators that are included in a network message source. For example, nurse Doe scans the bar code on an infusion pump added to introduce a pain reduction medication to patient Smith's IV tube. A device ID number represented in the bar code is designated herein for convenience as EID1. As a result, a message is sent over network 102 to alarm integration server 180 that ID reader 160b is associated with EID1. This also associates the patient PIDSMITH, (whose ID structure is most recently read) and the primary caregiver CIDDOE, with equipment EID1. This also indirectly associates nurse Doe's PDA that has a MAC address MAC1 with equipment EID1. If no new equipment is to be connected to the patient, step 352 is omitted. Control passes to step 354.

In step 354, the ID reader is used by the caregiver to read an ID structure attached to the container of medication that the caregiver is newly administering to the patient. The server 180 uses this information to associate the medication with the patient whose ID structure is most recently read. If the medication is administered using a piece of equipment, such as an infusion pump, the server 180 uses this information to associate the medication with the equipment whose ID structures is most recently read. For example, nurse Doe scans the bar code on fluid bag prepared by the pharmacy with properly diluted painkiller when she introduces the pain reduction medication to patient Smith's IV tube. The medication ID number represented in the bar code is designated herein for convenience as MID1. As a result, a message is sent over network 102 to alarm integration server 180 that ID reader 160b is associated with medication MID1. This also associates the most recently read infusion pump EID1, and patient PIDSMITH, and the primary caregiver CIDDOE with medication MID1. This also indirectly associates nurse Doe's PDA that has a MAC address MAC1 with medication MID1. If no new medication is to be connected to the patient, step 354 is omitted. Control passes to step 356.

In step 356, the ID reader is used by the caregiver to read an ID structure attached to equipment or a container of medication that is already associated with this same patient. The server 180 uses this information to disassociate the equipment or medication from the patient whose ID structure is most recently read. For example, nurse Doe scans the bar code on depleted fluid bag prepared by the pharmacy with properly diluted painkiller to remove this pain reduction medication from patient Smith's IV tube. The medication ID number represented in the bar code is designated herein for convenience as MID2. As a result, a message is sent over network 102 to alarm integration server 180 that ID reader 160b is disassociating with medication MID2. If equipment is involved, this makes that equipment the current equipment (equivalent to the equipment whose ID structure is most recently read). For example this makes infusion pump EID1 the current equipment. If no medication or equipment is to be removed from the patient, step 356 is omitted. Control passes back to step 350 to determine if any other changes are occurring for the current patient.

5. Method of Processing at an Alert Integration Server

FIG. 4A is a flow diagram that illustrates at a high level a method 400 for integrating messages from multiple diverse systems at an alert integration server 180 on the network, according to an embodiment.

In step 430, the server receives and archives association data. For example, the server 180 receives data from ID reader 160b operated by nurse Doe, as described above in step 330. More detailed steps on receiving and archiving the association data at the server 180 are described below with reference to FIGS. 4B and 4C. FIG. 4B is a flow diagram that illustrates in more detail step 430 in the method 400 for receiving and archiving association data, according to an embodiment. FIG. 4C is a block diagram that illustrates an association table 410 for holding in memory, or for storing or archiving association data, according to an embodiment.

Table 410 depicted in FIG. 4C organizes association data in rows and columns. In various embodiments, Table 410 is implemented on server 180 as a data structure in fast, volatile memory, or on slower non volatile storage media, described in more detail in a later section, or both. In the illustrated embodiment, Table 410 is implemented as a data structure in memory and the columns correspond to fields 412, 414, 416, 418, 420, 422, 424. The fields include a data-time field 412, a scanner ID field, a caregiver ID field, a patient ID field, a room number field 420, an equipment ID field 422, and a medication ID field. In other embodiments, more or fewer or different fields correspond to columns in an association table. The table 410 includes multiple rows that correspond to data records that are added to the table as the server 180 receives messages from one or more ID readers 160. Three records 428a, 428b, 428c, collectively referenced herein as records 428, are depicted in FIG. 3. Depending on the number of messages received and a policy for removing old records from the table, at other times or in other embodiments, Table 410 includes more or fewer rows. Table 410 is used to illustrate the accumulation of association data during step 430 described in more detail next, with respect to FIG. 4B.

In step 432, the server 180 receives from an ID reader 160 a message that includes a caregiver ID read by the reader 160. The network TCP/IP protocol includes the network address of the source of the message, the ID reader 160. The IDs of the caregiver and reader are associated in the server 180 by storing them in the same record in an association table 410 in memory used by server 180.

In some embodiments, the IP network address of ID reader 160 is used as the ID for the reader, but this address can change as the ID reader 160 connects to the network 102 at different wireless access points 110. In the illustrated embodiment, the MAC address of the ID reader 160, which does not change, is used as the ID for a reader 160.

For example, server 180 receives, from bar code scanner 160b, a message that includes the value CIDDOE, as described above in step 332. In response to receiving a message from bar code scanner 160b, indicated by the MAC address MAC2 associated with the IP source, the server 180 stores the value MAC2 in scanner field 414 of record 428a in association table 410. The server 180 determines that the value CIDDOE is a caregiver ID, and stores that value in caregiver ID field 416 in the same record 428a. Thus the caregiver CIDDOE and the bar code scanner MAC2 are associated in record 428a of association table 410.

These associations are dynamic and change over time. Therefore, in an illustrated embodiment, the server also stores in the data-time field 412 in record 428a data indicating the date and time when the message was received.

In some embodiments, before storing the value CIDDOE in record 428a, the server checks the value against a list of allowed values for caregivers in the facility obtained, for example, from the staffing server 130. If the value is not valid, the server 180 does not store the value in a record in Table 410. Instead the server 180 sends an error code back to the ID reader, using, for example, the IP source address or the MAC address of the ID reader which sent the message. Thus, the system is protected from using the ID reader 160 to scan a bar code of an irrelevant object, such as the bar code on some grocery item.

In some embodiments, step 432 includes determining the portable communications device to use in communicating with the caregiver, e.g, the MAC address MAC1 of the PDA assigned to nurse Doe. For example, this is done by requesting the MAC address of the PDA assigned to nurse Doe from the staffing server 130. In some embodiments the value MAC1 is stored in a portable communications device field (not shown) in table 410 or as part of caregiver ID field 416.

In step 434, the server 180 receives from an ID reader 160 a message that includes a patient ID read by the reader 160. The IDs of the caregiver and patient are associated in the server 180 by storing them in the same record in the association table. For example, server 180 receives from bar code scanner 160b a message that includes the value PIDSMITH, as described above in step 334. In response, the server 180 determines that the value PIDSMITH is a patient ID, and stores that value in patient ID field 418 in a record of Table 410. In some embodiments, the server 180 also stores a location ID in room number field 420; the location ID stored in field 420 is taken from field 420 in the most recent previous record with the same patient ID, e.g., PIDSMITH in field 418.

In some embodiments, the server 180 generates a new record in the association table and stores the new data and time in the new record along with the most recently received scanner ID and caregiver ID. For example, record 428b is written with the new date and time in the date-time field 412, MAC2 in the scanner ID field 414, CIDDOE in the caregiver field 416 and PIDSMITH in the patient ID field 418. An advantage of this embodiment is that an audit trail is created for the sequence of steps taken in the care of the patient. In some embodiments, the audit trail is written to nonvolatile storage, as described below during step 458.

In some embodiments, the new patient ID information is simply written to the most recent record that has a blank (null value) in the patient ID field 418. For example, the new time is written into the date-time field 412 and the value PIDSMITH is written to the patient ID field 418 of existing record 428a. In either case, the caregiver CIDDOE and the bar code scanner MAC2 are associated in association table 410. An advantage of these embodiments is that the association table remains smaller and easier to manage. In the illustrated embodiment, the records in the association table in memory are overwritten, but the original records are written to nonvolatile storage to preserve an audit trail.

In some embodiments, before storing the value PIDSMITH in record 428a or 428b, the server checks the value against a list of allowed values for patients in the facility obtained, for example, from the ADT server 120. If the value is not valid, the server 180 does not store the value in a record in Table 410. Instead the server 180 sends an error code back to the ID reader or to the portable communications device assigned to the caregiver. For example, the server sends an error message to MAC1, the PDA assigned to nurse Jones.

In some embodiments, step 434 includes determining whether the caregiver is the primary caregiver for the patient, for example, by requesting information from the staffing server 130 or ADT server 120 or both. If it is determined that the caregiver is the primary caregiver then a primary caregiver flag (not shown) within the caregiver ID field 416 is set.

In step 440, the server 180 receives from an ID reader 160 or portable communications device 150 a message that indicates the patient is being moved to a room. In some embodiments, step 440 includes a location ID read by the reader 160. In some embodiments, during step 440 the server 180 receives a solicited or unsolicited message from the ADT server 120 that indicates the patient is placed in a room indicated by a location ID. The IDs of the location and patient are associated in the server 180 by storing them in the same record in the association table. For example, server 180 receives from bar code scanner 160b a message that includes the value LID222, as described above in step 340. In response, the server 180 determines that the value LID222 is a location ID, and stores that value in room number field 420 in a record of Table 410, e.g., in record 428a. In some embodiments, before storing the value LID222 in record 428a or subsequent record, the server checks the value against a list of allowed values for that patient in the facility obtained, for example, from the ADT server 120. If the value is not valid, the server 180 does not store the value in Table 410, but sends an error code back to the caregiver. In some embodiments, step 440 is omitted.

In step 442, the server 180 receives from an ID reader 160 or portable communications device 150 a message that indicates a caregiver is the primary caregiver. In some embodiments, step 442 includes the same caregiver ID read by the reader 160 for a second time. For example, server 180 receives from bar code scanner 160b a message that includes the value CIDDOE, as described above in step 342. In response, the server 180 determines that the value CIDDOE is a repeat value and sets the primary caregiver flag in the caregiver ID field 416. In some embodiments, step 442 is omitted.

In step 450 it is determined if any equipment ID or medication ID is being received for the patient. For example, it is determined whether a message is received from the ID reader 160b within a reasonable time. If not, control passes to step 458.

In step 458, any changes in the association table data structure in memory to be archived, such as new records for an audit trail, are written to non-volatile storage. Control then passes to step 460 shown in FIG. 4A, and described below. In some embodiments, medical records for the patient are updated during step 458. For example, in some embodiments at least some association data 182 is sent to the medical records server 124 by the alert integration server 180. In some embodiments, equipment inventory 140 is updated during step 458 with information about the most recent location for the equipment.

If it is determined that an equipment ID or medication ID is received in the next message from the ID reader, then control passes to step 452. If a message with equipment ID is not received, step 452 is omitted, and control passes to step 454.

In step 452, the server 180 receives from an ID reader 160 a message that includes equipment ID read by the reader 160. The IDs of the equipment, caregiver and patient are associated in the server 180 by storing them in the same record in the association table. For example, server 180 receives from bar code scanner 160b a message that includes the value EID1, as described above in step 352. In response, the server 180 determines that the value is a valid equipment ID, for example using a message from the inventory server 140. The server then determines, whether the value already appears in the equipment ID field 422 of another record with the same patient with no intervening disassociation.

If the equipment ID is not already associated with this patient, then it is being added to this patient, and the server 180 stores that value in equipment ID field 422 in a record of Table 410 with a new time in the date-time field and with the most recent reader, caregiver, patient, and room IDs indicated in fields 414, 416, 418, 420, respectively.

If the equipment ID is currently associated with this patient, the message indicates that the equipment is being removed from use with this patient, and the disassociation is recorded in the association table. Any method can be used to disassociate the equipment. In some embodiments, the record that includes both the patient and the equipment is deleted from the association table. In a table used as an audit trail, a new record is written with a new time in the date-time field, the most recent reader, caregiver, patient, and room IDs indicated in fields 414, 416, 418, 420, respectively, and with the equipment ID written into the equipment ID field 422. In some embodiments a disassociation flag (not shown) in the equipment ID field 422 is set.

In step 454, the server 180 receives from an ID reader 160 a message that includes a medication ID read by the reader 160. The IDs of the medication, caregiver, patient, and equipment, if applicable, are associated in the server 180 by storing them in the same record in the association table. For example, server 180 receives from bar code scanner 160b a message that includes the value MID1, as described above in step 354. In response, the server 180 determines that the value is a valid medication ID, for example using a message from the inventory server 140. The server then determines whether the value already appears in the medication ID field 424 of another record with the same patient with no intervening disassociation.

If the medication ID is not already associated with this patient, then it is being added to this patient, and the server 180 stores that value in medication ID field 424 in a record of Table 410 with a new time in the date-time field and with the most recent reader, caregiver, patient, room, and equipment IDs indicated in fields 414, 416, 418, 420, 422, respectively.

If the medication ID is currently associated with this patient, the medication is consumed or being removed from use with this patient, and the disassociation is recorded in the association table. Any method can be used to disassociate the medication. In some embodiments, the record that includes both the patient and the medication is deleted from the association table. In a table used as an audit trail, a new record is written with a new time in the date-time field, the most recent reader, caregiver, patient, room and equipment IDs indicated in fields 414, 416, 418, 420, 422, respectively, and with the medication ID written into the medication ID field 4242. In some embodiments a disassociation flag (not shown) in the medication ID field 424 is set.

As a result of step 430, an association table 410 in memory used by server 180, and in some embodiments an audit trail written to non-volatile storage, hold data that is used to associate messages from medical data generating equipment with a primary caregiver for a patient.

Referring again to FIG. 4A, control passes from step 430 to step 460. In step 460, the alert integration server 180 receives one or more medical alert messages from one or more network message sources based on output from one or more medical data generators. For example, it is assumed for purposes of illustration that medical data generator 212 in network message source 210 depicted in FIG. 2A is an infusion pump, that the ID structure 280b attached to it indicates value EID1, and that device server 218 is a medication and pump server. A RS232 data port of the infusion pump is connected to the network 102 through network bridge 216. When the infusion pump detects a loss of pressure due to an empty fluid supply bag, for example, a pump message is sent to server 218 over network 102. In some embodiments, alarm integration server 180 detects the pump message as a medical alert message on the network. In some embodiments, the server 218 receives the pump message and server 180 polls the server 218 to determine if any pump messages indicating a loss of pressure have been received. In some embodiments, device server 218 automatically forwards a message to server 180 based on the pump message.

In step 462, the server 180 determines the portable communication device of the primary caregiver who should respond to the alerts using the association data. It is assumed, for purposes of illustration, that the alert message received by server 180 indicates a serial number for the infusion pump that detected the problem. It is further assumed that server 180 queries inventory server 140 to determine the medical facility equipment ID that corresponds to the serial number and receives a return message that the pump in question has equipment ID EID1. During step 462, the alert integration server 180 uses the association table 410 to determine that the equipment with EID1 appears most recently in record 428a. In record 428a the caregiver is CIDDOE and is primary, the patient is PIDSMITH, the room number is LID222, and the medication is MID1. It is further assumed that server 180 queries staffing server 130 to determine the PDA assigned to CIDDOE has MAC address MAC1. Thus the portable comminations device for the primary caregiver is determined to have address MAC1.

In step 470, the server 180 generates a caregiver message based on the alert messages. For example, in some embodiments, the caregiver message indicates that the pump with the given serial number is experiencing abnormally low pressure. Control then passes to step 480.

In some embodiments, the caregiver message generated in step 470 is also based on the association data as well. For example, in an illustrated embodiment, the caregiver message includes data that indicates the patient is PIDSMITH and the location is LID222 where the pump is experiencing abnormally low pressure.

In some embodiments, step 470 includes the steps 472, 474 and 476. In step 472, the server receives medical records for the patient. For example, the server queries the medical records server 124 to determine that the full name of patient PIDSMITH is John Smith and that the condition being treated is a broken femur. In some embodiments, the server 180 includes the full name and condition being treated in the caregiver message based on information in the medical records.

In step 474 the server 180 determines the priority and importance of the alert message. For purposes of illustration it is assumed that MID1 indicates a pain reduction medication. The server 180 determines that MID1 is a pain reduction medication using a medication inventory server such as inventory server 140. In some embodiments, the server 180 determines dose data that indicates the dose of the medication being administered based on pump operation status in the first alert message and association data that indicates the medication being administered. In some of these embodiments, the caregiver message includes the dose data.

In this example, the loss of pain reduction medication is serious for a broken leg, but not life threatening for short periods of time; and the server 180 determines that the loss of pump pressure is therefore of medium importance. The alert therefore is of lower priority than alerts that involve critical conditions, such as an overdose of a high-alert medication. Thus, in some embodiments, the caregiver message is based on multiple alert messages involving the same or multiple different patients.

In step 476, the server determines whether the caregiver message should be sent at the current time or at all. For example, a caregiver message based on a medium importance condition would not be warranted when the same caregiver is also primary for a patient suffering a higher priority or critical condition. As another example, a caregiver message based on an alert initiated from a machine that is no longer associated with any patient can be ignored altogether. In these cases control passes to step 498, described below.

It is further assumed for purposes of illustration that no other alerts of higher priority involve the same caregiver. In this case, control passes to step 480.

In step 480, the caregiver message is sent to the primary caregiver. For example, the caregiver message is sent to MAC address MAC1, the PDA of nurse Jane Doe, as determined in step 462. In the example the caregiver message is displayed on the PDA and indicates a low pressure pump alarm has been issued for a pump EID1 delivering pain reduction medication MID1 in room 222 where patient John Smith has a broken femur, and that this message is of medium importance.

In step 490, treatment is determined based on the alert or caregiver message. The treatment is indicted in a treatment message that is sent to a treatment device on the network capable of delivering the treatment. In many embodiments, the treatment device is also a medical data generator, such as an infusion pump. For example, in step 490 it is determined from the association data that a second infusion pump with equipment ID EID2 is associated with patient PIDSMITH and is loaded with another pain reduction medication MID3. During step 490, a treatment message is generated to increase the infusion rate of pump EID2. Using the inventory server 140, it is determined that pump EID2 has a MAC address MAC3. Based on the MAC address, network 102 sends the treatment message to EID2. As a result, pump EID2 increases the infusion rate of the medication MID3 into patient PIDSMITH. In some embodiments, step 490 is determined automatically by server 180. In an illustrated embodiment, the primary caregiver, nurse Doe, enters information as data on her PDA that is used to determine the treatment and form the treatment message. In some embodiments, step 490 is omitted. Control passes to step 498.

In step 498, location of equipment in the facility is determined based on the association data. For example, the most recent location for the equipment is sent to the equipment inventory server for storage during step 458, as described above. A facility staff member looking for a particular piece of equipment can then ascertain from the inventory server 140 where it was last used. Similarly, if stored in an audit trail described above, a staff member looking for a particular piece of equipment can search the audit trail to find the location in a record where the equipment was most recently disassociated.

6. Example Embodiment

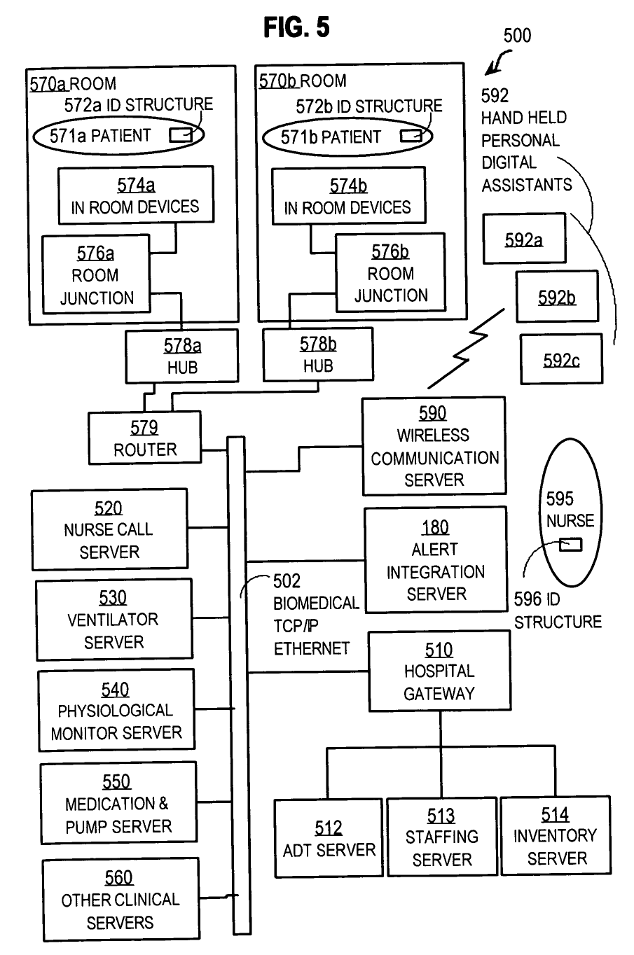

FIG. 5 is a block diagram that illustrates a system 500 for integrating medical messages from multiple diverse systems, according to a more detailed embodiment. System 500 includes biomedical TCP/IP Ethernet 502 connecting an alert integration server 180 with a hospital gateway 510, a nurse call server 520, ventilator server 530, physiological monitor server 540, medication and pump server 550, other clinical servers 560, a wireless communication server 590, and a router 579. The system also includes hand held personal digital assistants (PDAs) 592 communicating with the wireless communication server 590.

The system 500 further includes in-room devices 574a, 574b connected to room junctions 576a, 576b in rooms 570a, 570b, respectively. The room junctions connect to router 579 through hubs 578a, 578b. System 500 includes many more in-room devices and room junctions in corresponding additional rooms connected to hubs 578a and 578b, but these are not shown in FIG. 5 in order to avoid obscuring the description of this embodiment. The system also includes ID structures, such as bar code markings, on the in-room devices (not shown) and ID structures 596 on a badge worn by nurse 595 and ID structures 572a, 572b worn by patients 571a, 571b in rooms 570a, 570b, respectively. ID structures worn by other nurses and patients are not shown in order to avoid obscuring the description of this embodiment.

The hospital gateway 510 connects the biomedical TCP/IP Ethernet 502 with a separate LAN that connects an ADT server 512, staffing server 513, inventory server 514 and a medical records server (not shown). These servers are similar to the servers 120, 130, 140, 124, respectively, described above.

The in-room devices 574a, 574b include multiple medical data generators, such as patient stations, ventilators, physiological monitors (including vital signs monitors), infusion pumps, and other clinical devices. Network message sources, as described above, are formed by combining these in-room devices with their corresponding servers. For example, patient stations in multiple rooms and a nurse call server 520 form one network message source; ventilators in multiple rooms and ventilator server 530 form a second network message source; physiological monitors in multiple rooms and physiological monitor server 540 form a third network message source; infusion pumps in multiple rooms and medication & pump server 550 form a fourth network message source. Other clinical devices and their corresponding one or more other clinical servers 560 form one or more additional network message sources.

The in-room devices communicate with their servers through a LAN made up of room junctions 576a, 576b and hubs 578a, 578b connected to router 579 which determines which traffic on that LAN is directed to servers on the Biomedical TCP/IP Ethernet. Only one link is depicted in FIG. 5 between the in-room devices and the room junction in each room to avoid obscuring the drawing. In general, there is one link per in-room device; but not all the links are via cable. Wireless links are used by some in-room devices.

In the illustrated embodiment, the wireless hand held PDAs 592 are equipped with screens to display text and video data, text entry interfaces, speakers, audio sensors, video sensors and ID readers.

In an example use of system 500, the following events occur in order.

1] patient 571a in room 570a activates a call button interfaced to a digital patient station 250 among the in-room devices 574a; in response a caregiver message is sent to PDA 592a held by a first nurse, e.g., nurse 595.

2] In room 570b, a toilet emergency switch is pulled that is interfaced to a different digital patient station 250 among the in-room devices 574b; in response a caregiver message is sent to PDA 592b held by a second nurse.

3] In a third room (not shown), an infusion pump issues an alarm; in response a caregiver message is sent to PDA 592a held by the first nurse.

4] A person in room 570b activates a code blue button interfaced to the digital patient station 250; in response a caregiver message is sent to three PDA 592a, 592b, 592c held by the first, second and third nurses, overriding the caregiver messages sent in 1] and 2] and 3].

5] In room 570b, a ventilator issues an alarm; in response a caregiver message is sent to PDA 592b held by the second nurse. The caregiver message indicates that the second nurse better attend to the ventilator alarm and let other nurses attend to the code blue, thus integrating information from several different alarms and different patients into a single caregiver message.

7. Computer Hardware Overview

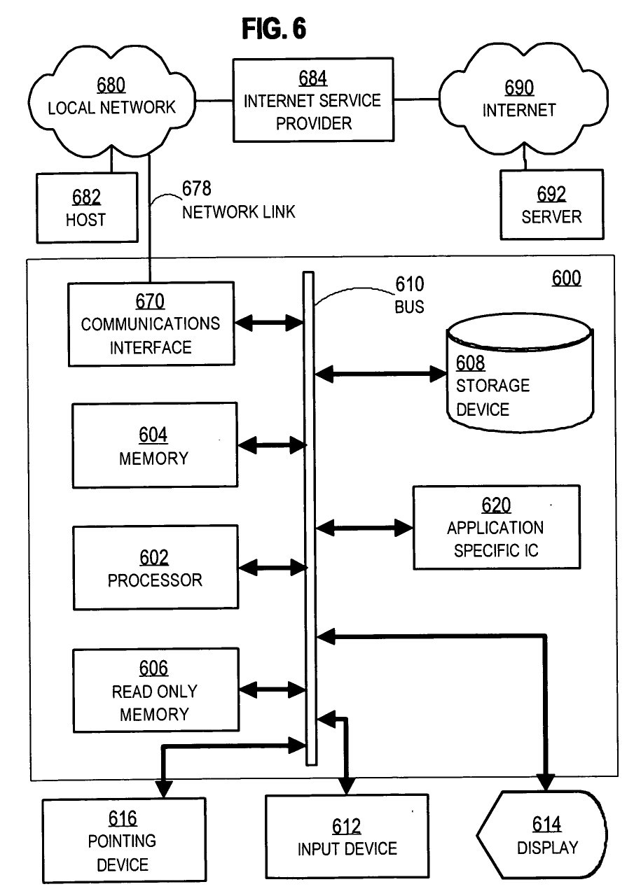

FIG. 6 is a block diagram that illustrates a computer system 600 upon which an embodiment of the invention may be implemented. Computer system 600 includes a communication mechanism such as a bus 610 for passing information between other internal and external components of the computer system 600. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit). A sequence of binary digits constitutes digital data that is used to represent a number or code for a character. A bus 610 includes many parallel conductors of information so that information is transferred quickly among devices coupled to the bus 610. One or more processors 602 for processing information are coupled with the bus 610. A processor 602 performs a set of operations on information. The set of operations include bringing information in from the bus 610 and placing information on the bus 610. The set of operations also typically include comparing two or more units of information, shifting positions of units of information, and combining two or more units of information, such as by addition or multiplication. A sequence of operations to be executed by the processor 602 constitute computer instructions.

Computer system 600 also includes a memory 604 coupled to bus 610. The memory 604, such as a random access memory (RAM) or other dynamic storage device, stores information including computer instructions. Dynamic memory allows information stored therein to be changed by the computer system 600. RAM allows a unit of information stored at a location called a memory address to be stored and retrieved independently of information at neighboring addresses. The memory 604 is also used by the processor 602 to store temporary values during execution of computer instructions. The computer system 600 also includes a read only memory (ROM) 606 or other static storage device coupled to the bus 610 for storing static information, including instructions, that is not changed by the computer system 600. Also coupled to bus 610 is a non-volatile (persistent) storage device 608, such as a magnetic disk or optical disk, for storing information, including instructions, that persists even when the computer system 600 is turned off or otherwise loses power.

Information, including instructions, is provided to the bus 610 for use by the processor from an external input device 612, such as a keyboard containing alphanumeric keys operated by a human user, or a sensor. A sensor detects conditions in its vicinity and transforms those detections into signals compatible with the signals used to represent information in computer system 600. Other external devices coupled to bus 610, used primarily for interacting with humans, include a display device 614, such as a cathode ray tube (CRT) or a liquid crystal display (LCD), for presenting images, and a pointing device 616, such as a mouse or a trackball or cursor direction keys, for controlling a position of a small cursor image presented on the display 614 and issuing commands associated with graphical elements presented on the display 614.

In the illustrated embodiment, special purpose hardware, such as an application specific integrated circuit (IC) 620, is coupled to bus 610. The special purpose hardware is configured to perform operations not performed by processor 602 quickly enough for special purposes. Examples of application specific ICs include graphics accelerator cards for generating images for display 614, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 600 also includes one or more instances of a communications interface 670 coupled to bus 610. Communication interface 670 provides a two-way communication coupling to a variety of external devices that operate with their own processors, such as printers, scanners and external disks. In general the coupling is with a network link 678 that is connected to a local network 680 to which a variety of external devices with their own processors are connected. For example, communication interface 670 may be a parallel port or a serial port or a universal serial bus (USB) port on a personal computer. In some embodiments, communications interface 670 is an integrated services digital network (ISDN) card or a digital subscriber line (DSL) card or a telephone modem that provides an information communication connection to a corresponding type of telephone line. In some embodiments, a communication interface 670 is a cable modem that converts signals on bus 610 into signals for a communication connection over a coaxial cable or into optical signals for a communication connection over a fiber optic cable. As another example, communications interface 670 may be a local area network (LAN) card to provide a data communication connection to a compatible LAN, such as Ethernet. Wireless links may also be implemented. For wireless links, the communications interface 670 sends and receives electrical, acoustic or electromagnetic signals, including infrared and optical signals, that carry information streams, such as digital data. Such signals are examples of carrier waves.

The term computer-readable medium is used herein to refer to any medium that participates in providing information to processor 602, including instructions for execution. Such a medium may take many forms, including, but not limited to, nonvolatile media, volatile media and transmission media. Nonvolatile media include, for example, optical or magnetic disks, such as storage device 608. Volatile media include, for example, dynamic memory 604. Transmission media include, for example, coaxial cables, copper wire, fiber optic cables, and waves that travel through space without wires or cables, such as acoustic waves and electromagnetic waves, including radio, optical and infrared waves. Signals that are transmitted over transmission media are herein called carrier waves.

Common forms of computer-readable media include, for example, a floppy disk, a flexible disk, a hard disk, a magnetic tape, or any other magnetic medium, a compact disk ROM (CAROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read.

Network link 678 typically provides information communication through one or more networks to other devices that use or process the information. For example, network link 678 may provide a connection through local network 680 to a host computer 682 or to equipment 684 operated by an Internet Service Provider (ISP). ISP equipment 684 in turn provides data communication services through the public, world-wide packet-switching communication network of networks now commonly referred to as the Internet 690. A computer called a server 692 connected to the Internet provides a service in response to information received over the Internet. For example, server 692 provides information representing video data for preservation at display 614.

The invention is related to the use of computer system 600 for implementing the techniques described herein. According to one embodiment of the invention, those techniques are performed by computer system 600 in response to processor 602 executing one or more sequences of one or more instructions contained in memory 604. Such instructions, also called software and program code, may be read into memory 604 from another computer-readable medium such as storage device 608. Execution of the sequences of instructions contained in memory 604 causes processor 602 to perform the method steps described herein. In alternative embodiments, hardware, such as application specific integrated circuit 620, may be used in place of or in combination with software to implement the invention. Thus, embodiments of the invention are not limited to any specific combination of hardware and software.

The signals transmitted over network link 678 and other networks through communications interface 670, which carry information to and from computer system 600, are exemplary forms of carrier waves. Computer system 600 can send and receive information, including program code, through the networks 680, 690 among others, through network link 678 and communications interface 670. In an example using the Internet 690, a server 692 transmits program code for a particular application, requested by a message sent from computer 600, through Internet 690, ISP equipment 684, local network 680 and communications interface 670. The received code may be executed by processor 602 as it is received, or may be stored in storage device 608 or other nonvolatile storage for later execution, or both. In this manner, computer system 600 may obtain application program code in the form of a carrier wave.

Various forms of computer readable media may be involved in carrying one or more sequence of instructions or data or both to processor 602 for execution. For example, instructions and data may initially be carried on a magnetic disk of a remote computer such as host 682. The remote computer loads the instructions and data into its dynamic memory and sends the instructions and data over a telephone line using a modem. A modem local to the computer system 600 receives the instructions and data on a telephone line and uses an infra-red transmitter to convert the instructions and data to an infra-red signal, a carrier wave serving as the network link 678. An infrared detector serving as communications interface 670 receives the instructions and data carried in the infrared signal and places information representing the instructions and data onto bus 610. Bus 610 carries the information to memory 604 from which processor 602 retrieves and executes the instructions using some of the data sent with the instructions. The instructions and data received in memory 604 may optionally be stored on storage device 608, either before or after execution by the processor 602.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense.

What is claimed is:

1. A method for integrating messages from a plurality of different medical care devices used to monitor or treat a patient, the method including the steps of:
receiving association data that indicates
an association between a particular patient and one portable communication device assigned to one primary medical caregiver, and
a plurality of associations between the patient and a corresponding plurality of medical data generators, wherein
a medical data generator is capable of generating medical data that supports a medical alert message that indicates attention is desired from a medical caregiver, and
the plurality of medical data generators direct the medical data to a plurality of different destinations;
determining whether a first medical alert message is issued based on medical data from any of the plurality of medical data generators; and
if it is determined that the first medical alert message is issued, then performing the steps of
determining the portable communication device based on the first medical alert message and the association data,
generating a caregiver message based at least in part on the first medical alert message, and
sending the caregiver message to the portable communication device.

2. The method as recited in claim 1 wherein the plurality of medical data generators includes at least one of a device for human communication, a device for monitoring a condition of a patient, and a device for delivering a treatment to a patient.

3. The method as recited in claim 1 wherein the plurality of medical data generators includes at least two of a device for human communication, a device for monitoring a condition of a patient, and a device for delivering a treatment to a patient.

4. The method as recited in claim 1 wherein the plurality of medical data generators includes a device for human communication, a device for monitoring a condition of a patient, and a device for delivering a treatment to a patient.

5. The method as recited in claim 2, wherein the device for human communication includes a manually operated switch that causes a medical alert message that indicates at least one of a nurse call, an emergency call, and a code blue call.

6. The method as recited in claim 2, wherein the device for human communication includes a voice sensor and causes a medical alert message that includes voice data.

7. The method as recited in claim 2, wherein the device for human communication includes a video camera and causes a medical alert message that includes video data.

8. The method as recited in claim 2, wherein the device for monitoring a condition of a patient is a vital signs monitor that causes a medical alert message that includes vital signs data.

9. The method as recited in claim 2, wherein the device for delivering a treatment to a patient is an infusion pump that causes a medical alert message that includes pump operation status.

10. The method as recited in claim 2, wherein the device for delivering a treatment to a patient is a lung ventilator that causes a medical alert message that includes ventilator operation status.

11. The method as recited in claim 2, wherein the device for delivering a treatment to a patient causes a medical alert message that indicates a reminder for the caregiver to perform a particular scheduled procedure.

12. The method as recited in claim 1, wherein:
the association data further indicates an association between the particular patient and a medication being administered to the particular patient; and
said step of generating the caregiver message further comprises generating the caregiver message based in part on the association data indicating the medication.

13. The method as recited in claim 12, wherein said medication is a highalert medication that has a high risk of causing injury when improperly administered.

14. The method as recited in claim 9, wherein:
the association data further indicates an association between the particular patient and a medication being administered to the particular patient by the infusion pump; and
said step of generating the caregiver message further comprises
determining dose data that indicates the dose of the medication being administered based on pump operation status in the first alert message and association data that indicates the medication being administered, and
generating the caregiver message based in part on the dose data.

15. The method as recited in claim 1, said step of generating the caregiver message further comprising:
receiving medical records data that indicates medical history of the particular patient; and
generating the caregiver message based in part on the medical records data.

16. The method as recited in claim 1, said step of generating the caregiver message further comprising:
determining a degree of importance for the first medical alert message; and
generating the caregiver message based in part on the degree of importance for the first medical alert message.

17. The method as recited in claim 16, wherein:
said step of generating the caregiver message further comprises determining whether the first medical alert message warrants a caregiver message based on the degree of importance; and
said step of sending the caregiver message further comprises sending the caregiver message only if it is determined that the first medical alert message warrants a caregiver message.

18. The method as recited in claim 1, said step of generating the caregiver message further comprising:
determining whether a second medical alert messages is issued based on medical data from any of the plurality of medical data generators within a limited time after the first medical alert message; and
if it is determined that the second medical alert messages is issued within the limited time, then generating the caregiver message based in part on the second medical alert message.

19. The method as recited in claim 1, wherein:
the association data further indicates
an association between a second patient, different from the particular patient, and the portable communication device, and
a second plurality of associations between the second patient and a corresponding second plurality of medical data generators; and
said step of generating the caregiver message further comprises
determining whether a second medical alert messages is issued based on medical data from any of the second plurality of medical data generators within a limited time after the first medical alert message, and
if it is determined that the second medical alert messages is issued within the limited time, then generating the caregiver message based in part on the second medical alert message.

20. The method as recited in claim 1, said step of receiving association data further comprising receiving first data and second data from a reader that senses an identification structure.

21. The method as recited in claim 20, wherein the identification structure is a bar code marking.

22. The method as recited in claim 20, wherein the identification structure is a radio frequency identification chip.

23. The method as recited in claim 20, said step of receiving first data and second data from the reader further comprising receiving:
first data that indicates a first identification structure connected to the primary medical caregiver; and
second data that indicates a second identification structure connected to the particular patient.

24. The method as recited in claim 20, said step of receiving first data and second data from the reader further comprising receiving:
first data that indicates a first identification structure connected to the particular patient; and
second data that indicates a second identification structure connected to a medical data generator of the plurality of medical data generators.

25. The method as recited in claim 20, said step of receiving first data and second data from the reader further comprising receiving:
first data that indicates a first identification structure connected to the particular patient; and
second data that indicates a second identification structure connected to a container for a medication that is being administered to the particular patient.

26. The method as recited in claim 1, further comprising the steps of:
determining a particular treatment to be performed by a particular device selected from the plurality of medical data generators, based at least in part on the first medical alert message, wherein said particular device delivers a treatment to a patient; and
sending to the particular device a treatment message that indicates the particular treatment to be performed, whereby the particular device delivers the particular treatment to the particular patient.

27. The method as recited in claim 26, said step of determining the particular treatment further comprising the step of receiving from the portable communications device an instructions message that indicates information entered by the primary medical caregiver for providing treatment to the particular patient.

28. The method as recited in claim 1, said step of receiving association data further comprising receiving association data that indicates a time when each association began.

29. The method as recited in claim 28, further comprising storing each association in the association data on a nonvolatile storage medium without removing association data previously stored on the storage medium, whereby an audit trail is produced for care given to the particular patient.

30. The method as recited in claim 1, wherein:
the association data further indicates an association between the particular patient and a particular location in a medical care facility; and
the method further comprises storing the particular location in association with each medical data generator of the plurality of medical data generators, whereby the plurality of medical data generators can be located in the medical care facility.

31. The method as recited in claim 1, said step of determining whether a first medical alert message is issued further comprising sampling message traffic on one or more communication channels to which the plurality of medical data generators direct the medical data.

32. The method as recited in claim 1, said step of determining whether a first medical alert message is issued further comprising requesting the first medical alert message from a particular server to which a type of generator among the plurality of medical data generators directs the medical data.

33. The method as recited in claim 1, said step of determining whether a first medical alert message is issued further comprising sampling message traffic on a network to which a network adapter directs captured data based on the medical data, wherein the network adapter is configured for capturing medical data from a medical data generator.

34. A system for integrating messages from a plurality of medical care devices used to monitor or treat a patient comprising:
a means for receiving association data that indicates
an association between a particular patient and one portable communication device assigned to one primary medical caregiver, and
a plurality of associations between the particular patient and a corresponding plurality of medical data generators, wherein
a medical data generator generates is capable of generating medical data that supports a medical alert message that indicates attention is desired from a medical care provider, and
the plurality of medical data generators direct the medical data to a plurality of different destinations;
a means for determining whether a first medical alert message is issued based on medical data from any of the plurality of medical data generators; and
a means for performing, if it is determined that the first medical alert message is issued, the steps of
determining the portable communication device based on the first medical alert message and the association data,
generating a caregiver message based at least in part on the first medical alert message, and
sending the caregiver message to the portable communication device.

35. A system for integrating messages from a plurality of medical care devices used to monitor or treat a patient comprising:
a network for communicating signals among a plurality of nodes connected to the network;
one portable communications device assigned to one primary medical caregiver and connected to the network;
a plurality of medical data generators connected to the network, wherein
a medical data generator is capable of generating medical data that supports a medical alert message that indicates attention is desired from a medical care provider, and
the plurality of medical data generators direct the medical data to a plurality of different destinations;
one or more processors connected to the network; and
one or more stored sequences of instructions which, when executed by the one or more processors, cause the one or more processors to carry out the steps of:
receiving association data that indicates an association between a particular patient and the portable communication device, and a plurality of associations between the particular patient and the plurality of medical data generators;
determining whether a first medical alert message is issued based on medical data from any of the plurality of medical data generators; and
if it is determined that the first medical alert message is issued, then performing the steps of
determining the portable communication device based on the first medical alert message and the association data,
generating a caregiver message based at least in part on the first medical alert message, and
sending the caregiver message to the portable communication device.

36. The system as recited in claim 35, wherein:
the system further comprises
a plurality of identification structures connected to a patient, a caregiver, and the plurality of medical data generators, and
a reader for sensing identification structures, wherein the reader is connected to the network; and
said step of receiving association data further comprises receiving a sequence of signals from the reader that indicates the association data.

37. A computer-readable medium carrying one or more sequences of instructions for integrating messages from a plurality of medical care devices used to monitor or treat a patient, wherein execution of the one or more sequences of instructions by one or more processors causes the one or more processors to perform the steps of:
receiving association data that indicates
an association between a particular patient and one portable communication device assigned to one primary medical caregiver, and
a plurality of associations between the particular patient and a corresponding plurality of medical data generators, wherein
a medical data generator is capable of generating medical data that supports a medical alert message that indicates attention is desired from a medical care provider, and
the plurality of medical data generators direct the medical data to a plurality of different destinations;
determining whether a first medical alert message is issued based on medical data from any of the plurality of medical data generators; and
if it is determined that the first medical alert message is issued, then performing the steps of
determining the portable communication device based on the first medical alert message and the association data,
generating a caregiver message based at least in part on the first medical alert message, and
sending the caregiver message to the portable communication device.

38. A apparatus for reporting human communications regarding the condition or treatment of a patient, the apparatus comprising:
a network interface that is coupled to a network for communicating therewith a data packet;
a plurality of switches for indicating multiple conditions in the vicinity of a patient;
a sound sensor; and
one or more circuit blocks configured to perform the steps of:
detecting closure of a first switch among the plurality of switches;
in response to detecting closure of the first switch, sending a first data packet that indicates a condition associated with the first switch to the network through the network interface;
forming digital sound data from sound sensed by the sound sensor;
forming a stream of data packets based on the digital sound data using a protocol for transferring sound over the network; and
sending the stream of data packets to the network through the network interface.
